(19) United States
(12) Patent Application Publication
(10) Pub. No.: US 2005/0070692 A1
(43) Pub. Date: Mar. 31, 2005

(54) Anti-interleukin-1 beta analogs

(76) Inventors: John Michael Beals, Indianapolis, IN (US); Lihua Huang, Carmel, IN (US); Jirong Lu, Vancouver, IN (US); Derrick Ryan Witcher, Fishers, IN (US)

Correspondence Address: ELI LILLY AND COMPANY;PATENT DIVISION, P.O. BOX 6288, INDIANAPOLIS, IN, 46206-6288, US

(21) Appl. No.: 10/503,504

(22) PCT Filed: Feb. 20, 2003
(86) PCT No.: PCT/US03/03117

Related U.S. Application Data
(60) Provisional application No. 60/361,423, filed on Feb. 28, 2002.

(51) Int. Cl. C07K 16/44

(52) U.S. Cl. 530/388.15

(57) ABSTRACT

The present invention encompasses analogs of humanized antibody Hu007 that neutralize IL-1β activity in vivo. These antibodies can be used to treat various diseases such as rheumatoid arthritis and osteoarthritis.

Interleukin-1β (IL-1β) is a proinflammatory cytokine. IL-1β over-production has been implicated in the pathogenesis of a variety of diseases such as rheumatoid arthritis and osteoarthritis. IL-1β has been shown to increase cell migration into the inflamed synovium of joints by the up-regulation of adhesion molecules, the stimulation of the production of prostaglandins and metalloproteinase, the inhibition of collagen and proteoglycan synthesis, and the stimulation of osteoclastic bone resorption. Because of these properties, IL-1 is one of the primary mediators of bone and cartilage destruction in arthritis. Thus, agents that reduce the activity of IL-1β represent possible treatments for diseases such as arthritis.

There are three members of the IL-1 gene family: IL-1α, IL-1β, and IL-1 receptor antagonist (IL-1ra). IL-1α and IL-1β are agonists of the IL-1 receptor whereas the IL-1ra is a specific receptor antagonist and thus, an endogenous competitive inhibitor of IL-1. Administration of recombinant IL-1ra to patients in clinical trials provided significant clinical improvements in patients with severe rheumatoid arthritis compared to placebo. Furthermore, administration of IL-1ra reduced the rate of progressive joint damage. However, the poor pharmacokinetic properties and the large dose that must be administered make recombinant IL-1ra a less than ideal therapeutic agent.

A high affinity neutralizing antibody to IL-1β would make a superior therapeutic agent. The typically long elimination half-lives of antibodies coupled with high affinity binding result in a therapeutic agent wherein much lower concentrations can be dosed much less frequently than recombinant IL-1ra. Although numerous IL-1β antibodies have been described, it has been exceedingly difficult to identify monoclonal antibodies having high affinity, high specificity, and potent neutralizing activity.

The present invention encompasses analogs of a high affinity humanized antibody directed against human IL-1β. These analogs are high affinity antibodies with improved stability that have potent IL-1β neutralizing activity and are highly specific for IL-1β.

It has been found that a deamidation site in the CDR2 region of the heavy chain influences the biological properties of a high affinity humanized antibody directed to human IL-1β. Analogs of this high affinity antibody slow down or eliminate deamidation which results in improved stability.

This invention encompasses analogs of Hu007 that specifically bind mature human IL-1β. The invention includes analogs in which deamidation is reduced or eliminated comprising at least one amino acid substitution at positions 54, 55 or 56 of the heavy chain complementarity determining region 2 (CDR2), SEQ ID NO: 1, Glu Ile Leu Pro Xaa₅₄ Xaa₅₅ Xaa₅₆ Asn Ile Asn Tyr Asn Gln Lys Phe Lys Gly (SEQ ID NO:1)

wherein:

Xaa at position 54 is Gly, Ala, Ser, Val, Thr, Leu, Ile, Met, Phe, Tyr, or Trp;
Xaa at position 55 is Asn, Gln, Arg, Asp, Ser, Gly, or Ala;
Xaa at position 56 is Gly, Ala, Ser, Val, Thr, Leu, Ile, Met, Phe, Tyr, or Trp;
provided that when Xaa₅₅ is Asn, Xaa₅₆ is not Gly.
   Preferred embodiments include the analogs wherein:
Xaa₅₄ is Gly, Xaa₅₅ is Asn, and Xaa₅₆ is Val;
Xaa₅₄ is Gly, Xaa₅₅ is Asn, and Xaa₅₆ is Ala;
Xaa₅₄ is Gly, Xaa₅₅ is Asp, and Xaa₅₆ is Gly;
Xaa₅₄ is Gly, Xaa₅₅ 55 is Gln, and Xaa₅₆ is Gly;
Xaa₅₄ is Gly, Xaa₅₅ 55 is Ala, and Xaa₅₆ is Gly;
Xaa₅₄ is Gly, Xaa₅₅ 55 is Gly, and Xaa₅₆ is Gly;
Xaa₅₄ is selected from the group consisting of Gly, Ala, Ser, Val, Thr, Leu, Ile, Met, Phe, Tyr, and Trp, Xaa₅₅ 55 is Ala, and Xaa₅₆ is Gly; and
Xaa₅₄ is selected from the group consisting of Gly, Ala, Ser, Val, Thr, Leu, Ile, Met, Phe, Tyr, and Trp, Xaa₅₅ 55 is Gly, and Xaa₅₆ is Gly.
   Most preferred is the analog wherein Xaa₅₄ is Gly, Xaa₅₅ is Ser, and Xaa₅₆ is Gly.

Another preferred analog comprises humanized antibody Hu007 which comprises a full length light chain corresponding to Formula I which is SEQ ID NO:7 and a full length heavy chain corresponding to Formula II which is SEQ ID NO:10, wherein said Formnula II contains the CDR2 region, SEQ ID NO:1. The analogs of the present invention include analogs having framework regions that have at least 65% identity with the corresponding framework regions in mouse monoclonal antibody Mu007.

It is also preferred that the analogs of the present invention have binding affinities within 10-fold that of mouse monoclonal antibody Mu007 or humanized antibody Hu007 and have potent neutralizing activity with IC50 values within 10-fold that of mouse monoclonal antibody Mu007 or humanized antibody Hu007.

The invention includes isolated nucleic acids comprising polynucleotides that encode the antibodies described and claimed herein. The invention also encompasses host cells transfected with these polynucleotides that express the antibodies described and claimed herein.

The invention encompasses methods of treating rheumatoid arthritis and osteoarthritis which comprise administering to a subject an effective amount of an antibody described and claimed herein as well as a method of inhibiting the destruction of cartilage that occurs in subjects that are prone to or have arthritis.

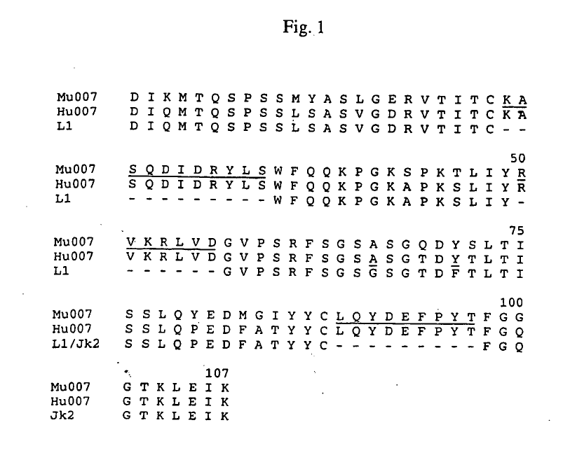

FIG. 1. Alignment of variable light chain amino acid sequences from Mu007, Hu007, and the germline L1 and Jκ2 segments. The CDR sequences based on the definition of Kabat are underlined in the Mu007 variable light chain sequence. The CDR sequences in the acceptor human variable light segment are omitted.

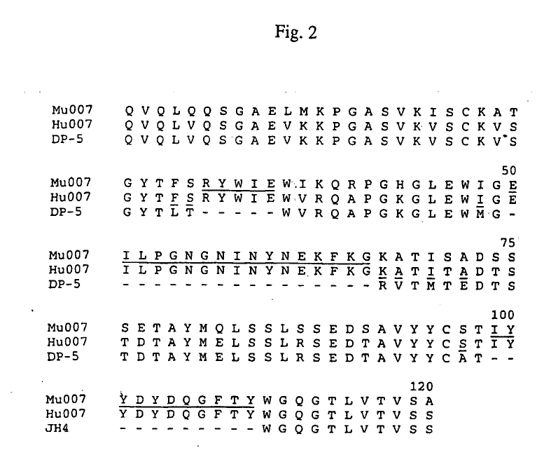

FIG. 2. Alignment of variable heavy chain amino acid sequences from Mu007, Hu007, and the germline DP5 and JH4 segments. The CDR sequences based on the definition of Kabat are underlined in the Mu007 variable heavy chain sequence. The CDR sequences in the acceptor human variable heavy segment are omitted.

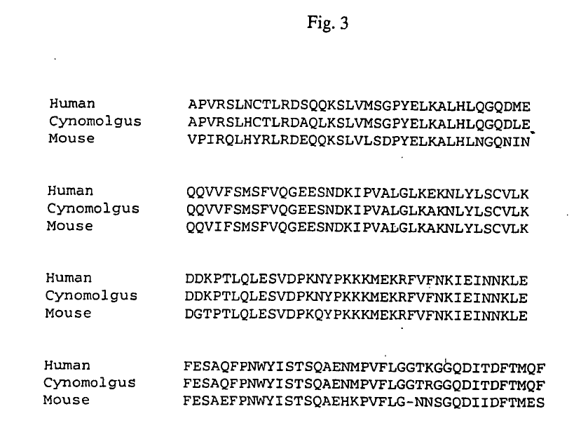

FIG. 3. Alignment of the mature IL-1β protein sequences from human, cynomolgous monkey, and mouse.

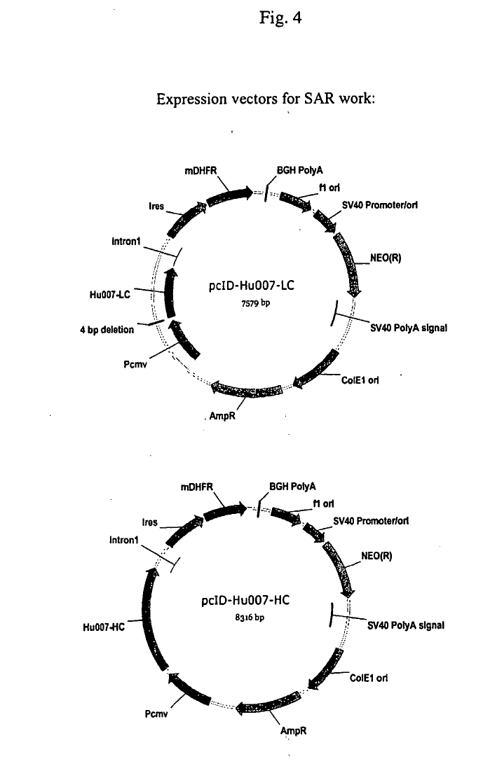

FIG. 4. Plasmid constructs for expression of Hu007 analogs. The Hu007 variable light and variable heavy genes were constructed as mini-exons.

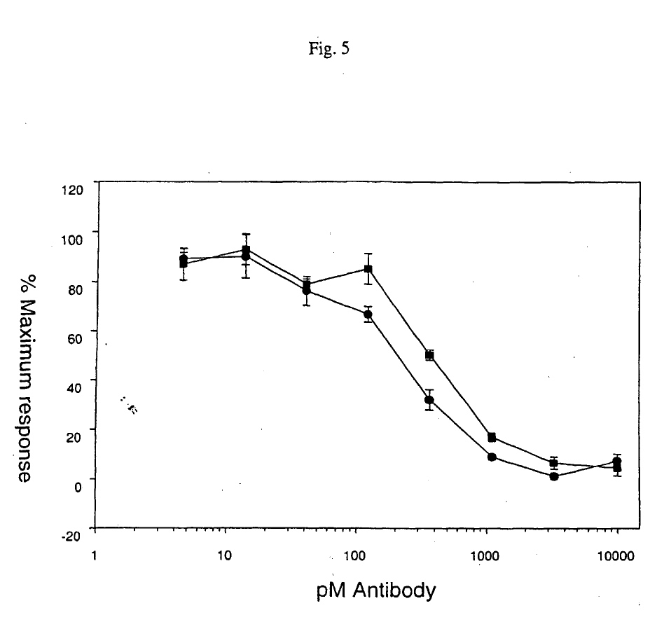

FIG. 5. Graph depicting the ability of Mu007 (●) and Hu007 (▪) to inhibit the proliferation of an IL-1β-dependent cell line.

The present invention encompasses analogs to Hu007, preferably humanized analogs, which bind the same epitope on human IL-1β as mouse monoclonal antibody Mu007 and humanized antibody Hu007. Preferably, these analogs are comprised of the heavy chain CDR2, SEQ ID NO:1, and the complementarity determining regions (CDRs) of the Mu007 antibody. The framework and other portions of these analogs may originate from a human germ line. The humanized versions of the Mu007 antibody retain the high affinity, high specificity, and potent neutralizing activity observed for the Mu007 murine antibody.

As used herein, the word “treat” includes therapeutic treatment, where a condition to be treated is already known to be present, and prophylaxis—i.e., prevention of, or amelioration of, the possible future onset of a condition. A “subject” means a mammal, preferably a human having need of treatment. Subjects having need of treatment include mammals that are prone to arthritis, mammals that exhibit any cartilage destruction, and mammals that have signs and symptoms associated with rheumatoid arthritis or osteoarthritis.

“Antibody” means a complete antibody molecule, having full length heavy and light chains; a fragment thereof, such as a F_{ab}, F_{ab}′, or F_{(ab′)2} or Fv fragment; a single chain antibody fragment, e.g. a single chain Fv, a heavy chain monomer or dimer; multivalent monospecific antigen binding proteins comprising two, three, four, or more antibodies or fragments thereof bound to each other by a connecting structure which binds the same epitope as mouse monoclonal antibody Mu007 or humanized antibody Hu007. In some contexts, herein, fragments will be mentioned specifically for emphasis; nevertheless, it will be understood that regardless of whether fragments are specified, the term “antibody” includes such fragments as well as single-chain forms. As long as the protein retains the ability to bind the same epitope on human ILIS as Mu007 or Hu007 and includes the heavy chain CDR2, SEQ ID NO: 1, it is included within the term “antibody.” Preferably, but not necessarily, the antibodies useful in the invention are produced recombinantly.

“Hu007” refers to a high affinity humanized antibody which binds the same epitope on human IL-1β as mouse-monoclonal antibody Mu007 (see U.S. provisional patent application Ser. No. 60/312,278).

The term “analog” refers to the Hu007 antibody which has at least one amino acid substitution which results in the reduction or elimination of the deamidation of an amino acid in a CDR region which in turn results in an antibody of increased stability. For example, analog refers to antibodies of the present invention which have at least one amino acid substitution at positions 54, 55, or 56 of the CDR2 region of the heavy chain which results in the reduction or elimination of the deamidation of position 55 of the heavy chain CDR2 region (Hu007 analogs).

Analogs that “specifically bind” mature human IL-1β (anti-IL-1β analogs) include analogs as defined above that bind the mature form of human IL-1β known in the art and represented in FIG. 3 and do not bind mature human IL-1α. An analog that specifically binds mature human IL-1β may show some cross-reactivity with mature IL-1β from other species.

The term “recombinant” in reference to an antibody includes antibodies that are prepared, expressed, created or isolated by recombinant means. Representative examples include antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see e.g., Taylor, L. D., et al., Nucl. Acids Res. 20:6287-6295,(1992); or antibodies prepared, expressed, created or isolated by any means that involves splicing of human immunoglobulin gene sequences to other DNA sequences. Such recombinant human antibodies have variable and constant regions derived from human germline immunoglobulin sequences.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function.

Light chains are classified as kappa and lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 12 or more amino acids, with the heavy chain also including a “D” region of about 3 or more amino acids.

IgG antibodies are the most abundant immunoglobulin in serum. IgG also has the longest half-life in serum of any immunoglobulin. Unlike other immunoglobulins, IgG is efficiently.recirculated following binding to FcRn. There are four IgG subclasses G1, G2, G3, and G4, each of which has different effector functions. G1, G2, and G3 can bind C1q and fix complement while G4 cannot. Even though G3 is able to bind C1q more efficiently than G1, G1 is more effective at mediating complement-directed cell lysis. G2 fixes complement very inefficiently. The C1q binding site in IgG is located at the fγ2 beta strand (amino acids 318-322), b6 bend (residue 331) and lower hinge (residues 235 and 237), which are also adjacent in three-dimensional space.

Human IgG4 exists in two molecular forms due to the heterogeneity of the inter-heavy chain disulfide bridges in the hinge region in a portion of secreted human IgG4 This heterogeneity is only revealed under denaturing, non-reducing conditions in which an HL “half-antibody” is detected (Angal, et al., Molecular Immunology 30(1):105 (1993)). (IgG4 hinge region sequence: ES-KYGPP - - - CPSCP, wherein the S is position 229 (the numbering is based on the N-linked glycosylation site at Asn 297 which is according to Kabat “Sequences of Proteins of Immunological Interest” National Institutes of Health, Bethesda, Md., 1987 and 1991). A mutation from S to P at position 241 in the IgG4 hinge region eliminates the half-antibody which leads to the production of a homogeneous antibody (Angal et al., 1993)

All IgG subclasses are capable of binding to Fc receptors (CD16, CD32, CD64) with G1 and G3 being more effective than G2 and G4. The Fc receptor-binding region of IgG is formed by residues located in both the hinge and the carboxy-terminal regions of the CH2 domain.

IgA can exist both in a monomeric and dimeric form held together by a J-chain. IgA is the second most abundant Ig in serum, but it has a half-life of only 6 days. IgA has three effector functions. It binds to an IgA specific receptor on macrophages and eosinophils, which drives phagocytosis and degranulation, respectively. It can also fix complement via an uiknown alternative pathway.

IgM is expressed as either a pentamer or a hexamer, both of which are held together by a J-chain. IgM has a serum half-life of 5 days. It binds weakly to C1q via a binding site located in its CH3 domain. IgD has a half-life of 3 days in serum. It is unclear what effector functions are attributable to this Ig. IgE is a monomeric Ig and has a serum half-life of 2.5 days. IgE binds to two Fc receptors, which drives degranulation and results in the release of proinflammatory agents.

Depending on the desired in vivo effect and the desired half-life, the antibodies of the present invention may contain any of the isotypes described above or may contain mutated regions wherein the complement and/or Fc receptor binding functions have been altered.

The variable regigns of each light/heavy chain pair form the antibody binding site.

Thus, an intact antibody has two binding sites. The chains all exhibit the same general structure of relatively conserved framework regions (FR) joined by three hypervariable regions, also called complementarity determining regions or CDRs. The framework regions align the CDRs from the two chains of each pair, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with well known conventions [Kabat, 1987 and 1991; Chothia, et al., J. Mol. Biol. 196:901-917 (1987); Chothia, et al., Nature 342:878-883 (1989)].

“Humanized antibody” means an antibody that is composed partially or fully of amino acid sequences derived from a human antibody germline or a rearranged sequence and made by altering the sequence of an antibody having non-human complementarity determining regions (CDR). The framework regions of the variable regions are substituted by corresponding human framework regions leaving the non-human CDR substantially intact. The framework region may be entirely human or may contain substitutions in regions that influence binding of the antibody to the target antigen. These regions may be substituted with the corresponding non-human amino acids. As discussed herein, antibody in the context of humanized antibody is not limited to a full-length antibody and can include fragments and single chain forms. Alternatively, it is recognized that the framework can be fixed to the human germline sequence and the non-human CDR domains can be inserted and the affinity matured through mutagenesis to mitigate any loss of affinity due to steric interactions between the non-human CDRs and the fully human framework.

Humanized antibodies have several potential advantages over non-human and chimeric antibodies for use in human therapy. For example, the human immune system should not recognize the framework or constant region of the humanized antibody as foreign, and therefore the antibody response against such an antibody should be less than against a totally foreign non-human antibody or a partially foreign chimeric antibody. In addition, parenterally-administered humanized antibodies generally have a longer half-life in the circulation than non-human antibodies. Furthermore, if effector functions are desired, because the effector portion is human, they may interact better with the other parts of the human immune system.

The term “deamidated or deamidation” refers to the degradation of Asn or Gin residues in a protein/peptide (Robinson, et al. (2001) Proc. Natl Acad. Sci. USA 12409-12413). For example, the intramolecular pathway for asparagine deamidation is via intermediate succinimide formation, resulting in a mixture of aspartyl and isoaspartyl residues (Harris, et al. (2001) J. of Chromatography 752:233-245). Deamidation may lead to a reduction of stability and/or the reduction or loss of activity of the protein. Deamidation can occur ex vivo during the preparation of the formulated therapeutic, negatively impacting the manufacturing and storage of the pharmaceutical agent. Moreover, the deamidation can occur in vivo effecting the antibody's efficacy and duration of action.

Preferably, the analogs of the present invention include analogs of Hu007 that specifically bind mature human IL-1β. The invention includes analogs in which deamidation is reduced or eliminated at position Asn55 by site specific changes, comprising at least one amino acid substitution at positions 54, 55 or 56 of the heavy chain complementarity determining region 2 (CDR2), SEQ ID NO:1, Glu Ile Leu Pro Xaa₅₄ Xaa₅₅ Xaa₅₆ Asn Ile Asn Tyr Asn Gln Lys Phe Lys Gly (SEQ ID NO:1)

wherein:,

Xaa at position 54 is Gly, Ala, Ser, Val, Thr, Leu, Ile, Met, Phe, Tyr, or Trp;
Xaa at position 55 is Asn, Gln, Arg, Asp, Ser, Gly, or Ala;
Xaa at position 56 is Gly, Ala, Ser, Val, Thr, Leu, Ile, Met, Phe, Tyr, or Trp.
   Preferred embodiments include the analogs wherein:
Xaa₅₄ is Gly, Xaa₅₅ is Asn, and Xaa₅₆ is Val;
Xaa₅₄ is Gly, Xaa₅₅ is Asn, and Xaa₅₆ is Ala;
Xaa₅₄ is Gly, Xaa₅₅ is Asp, and Xaa₅₆ is Gly;
Xaa₅₄ is Gly, Xaa₅₅ 55 is Gin, and Xaa₅₆ is Gly;
Xaa₅₄ is Gly, Xaa₅₅ 55 is Ala, and Xaa₅₆ is Gly;
Xaa₅₄ is Gly, Xaa₅₅ 55 is Gly, and Xaa₅₆ is Gly;
Xaa₅₄ is is selected from the group consisting of Gly, Ala, Ser, Val, Thr, Leu, Ile, Met, Phe, Tyr, and Trp, Xaa₅₅ 55 is Ala, and Xaa₅₆ is Gly; and
Xaa₅₄ is selected from the group consisting of Gly, Ala, Ser, Val, Thr, Leu, Ile, Met, Phe, Tyr, and Trp, Xaa₅₅ 55 is Gly, and Xaa₅₆ is Gly.
   Most preferred is the analog wherein Xaa₅₄ is Gly, Xaa₅₅ is Ser, and Xaa₅₆ is Gly.

The preferred analogs of the present invention have binding specificity, binding affinity, and potency similar to that observed for Mu007. The properties that define the analogs of the present invention reside primarily in the variable regions of the antibody. Thus, the complete light chain and heavy chain variable regions of the Mu007 antibody can be used in the context of any constant region, and the binding affinity and specificity as well as ability to neutralize mature human IL-1β will be generally unaffected. “Mu007” as used herein refers to the variable chain sequences represented in FIGS. 1 and 2 in the context of any mouse constant region, preferably a kappa light chain and a gamma-1 heavy chain.

A preferred analog of the present invention is a humanized antibody comprised of the heavy chain CDR2, SEQ ID NO:1 and one or more CDRs with the following amino acid sequences:

| | |
|---|---|
| Light Chain CDR1: | |
| Lys Ala Ser Gln Asp Ile Asp Arg Tyr Leu | SEQ ID NO:2 |
| Ser | |
| Light Chain CDR2: | |
| Arg Val Lys Arg Leu Val Asp | SEQ ID NO:3 |
| Light Chain CDR3: | |
| Leu Gln Tyr Asp Glu Phe Tyr Thr | SEQ ID NO:4 |
| Heavy Chain CDR1: | |
| Arg Tyr Trp Ile Glu | SEQ ID NO:5 |
| Heavy Chain CDR3: | |
| Ile Tyr Tyr Asp Tyr Asp Gln Gly Phe Thr | SEQ ID NO:6 |
| Tyr | |

In principle, a framework sequence from any human antibody may serve as the template for CDR grafting. However, straight chain replacement onto such a framework often leads to some loss of binding affinity to the antigen. The more homologous a human antibody is to the original murine antibody, the less likely the possibility that combining the murine CDRs with the human framework will introduce distortions in the

CDRs that could reduce affinity. Therefore, it is preferable that the human variable-region framework that is chosen to replace the murine variable-region framework apart from the CDRs has at least a 65% sequence identity with the murine antibody variable-region framework. It is more preferable that the human and murine variable regions apart from the CDRs have at least 70% sequence identify. It is even more preferable that the human and murine variable regions apart from the CDRs have at least 75% sequence identity. It is most preferable that the human and murine variable regions apart from the CDRs have at least 80% sequence identity. For example, a preferred human framework region for the variable light chain of the antibodies of the present invention as shown in FIG. 1 has approximately 80% sequence identity with the corresponding mouse sequence outside the CDRs. A preferred human framework region for the variable heavy chain of the antibodies of the present invention as shown in FIG. 2 has approximately 70% sequence identity with the corresponding mouse sequence outside the CDRs.

The heavy and light chain variable region framework residues can be derived from the same or different human antibody sequences. The human antibody sequences can be the sequences of naturally occurring human antibodies or can be consensus sequences of several human antibodies. Preferred human framework sequences for the heavy chain variable region of the humanized antibodies of the present invention include the VH segment DP-5 (Tomlinson, et al. (1992) J. Mol. Biol. 227:776-798) and the J segment JH4 (Ravetch, et al. (1981) Cell 27:583-591). The Vk segment L1 (Cox, et al. (1994) Eur. J. Immunol. 24:827-836) and the J segment Jk2 (Hieter, et al. (1982) J. Biol. Chem. 10:1516-1522) are preferred sequences to provide the framework for the humanized light chain variable region.

Certain amino acids from the human variable region framework residues were substituted with the corresponding murine amino acid to minimize effects on CDR conformation and/or binding to the IL-1β antigen.

Generally, when an amino acid falls under the following category, the framework amino acid of a human immunoglobulin to be used (acceptor immunoglobulin) is replaced by a framework amino acid from a CDR-providing non-human immunoglobulin (donor immunoglobulin):
(a) the amino acid in the human framework region of the acceptor immunoglobulin is unusual for human immunoglobulin at that position, whereas the corresponding amino acid in the donor immunoglobulin is typical for human immunoglobulin at that position;
(b) the position of the amino acid is immediately adjacent to one of the CDRs; or
(c) any side chain atom of a framework amino acid is within about 5-6 angstroms (center-to-center) of any atom of a CDR amino acid in a three dimensional immunoglobulin model [Queen, et al., Proc. Natl Acad. Sci. USA 86:10029-10033 (1989), and Co, et al., Proc. Natl. Acad. Sci. USA 88, 2869(1991)]. When each of the amino acids in the human framework region of the acceptor immunoglobulin and a corresponding amino acid in the donor immunoglobulin is unusual for human immunoglobulin at that position, such an amino acid is replaced by an amino acid typical for human immunoglobulin at that position.

Analysis of the preferred framework regions for the humanized antibodies of the present invention suggested several amino acids that may have significant contact with the CDRs. These amino acids from mouse monoclonal antibody Mu007 were substituted for the original human framework amino acids.

FIGS. 1 and 2 provide an alignment of the variable light and heavy regions from the mouse sequence, a preferred humanized sequence, and a preferred human germline sequence. The single underlined amino acids in the humanized sequence were substituted with the corresponding mouse residues. For example, this was done at residues 29, 30, 48, 67, 68, 70, 72 and 97 of the heavy chain. For the light chain, the replacements were made at residues 66 and 71.

The primary impetus for humanizing antibodies from another species is to reduce the possibility that the antibody causes an immune response when injected into a human patient as a therapeutic. The more human sequences that are employed in a humanized antibody, the lower the risk of immunogenicity. Changes can be made to the sequences described herein as preferable heavy and light chain regions without significantly affecting the biological properties of the antibody. This is especially true for the antibody constant regions and parts of the variable region which do not influence the ability of the CDRs to bind to IL-1β.

Furthermore, as discussed herein other human framework variable regions and variants thereof may be used in the present invention. However, regardless of the framework chosen, if reducing the risk of immunogenicity is a focus, the number of changes relative to the human framework chosen should be minimized.

A preferred light chain variable region for the antibodies of the present invention comprises Formula I which is SEQ ID NO:7. The CDRs based on the definition of Kabat are underlined.

1 2 3 4 5 6 7 8 9 10 11 12 13 14 15
Asp Ile Xaa Met Thr Gln Xaa Pro Ser Ser Xaa Xaa Ala Ser Xaa
16 17 18 19 20 21 22 23 24 25 26 27 28 29 30
Gly Xaa Arg Val Thr Ile Thr Cys Lys Ala Ser Gln Asp Ile Asp
31 32 33 34 35 36 37 38 39 40 41 42 43 44 45
Arg Tyr Leu Ser Trp Phe Gln Gln Lys Pro Gly Lys Ala Pro Lys
46 47 48 49 50 51 52 53 54 55 56 57 58 59 60
Xaa Leu Ile Tyr Arg Val Lys Arg Leu Val Asp Gly Val Pr Ser
61 62 63 64 65 66 67 68 69 70 71 72 73 74 75
Arg Phe Ser Gly Ser Xaa Ser Gly Xaa Asp Tyr Thr Leu Thr Ile
76 77 78 79 80 81 82 83 84 85 86 87 88 89 90
Ser Ser Leu Gln Pro Glu Asp Phe Ala Thr Tyr Tyr Cys Leu Gln
91 92 93 94 95 96 97 98 99 100 101 102 103 104 105 106 107
Tyr Asp Glu Phe Pro Tyr Thr Phe Gly Gln Gly Thr Lys Leu Glu Ile Lys

Xaa at position 3 is Gln or Lys;
Xaa at position 7 is Ser or Thr;
Xaa at position 11 is Leu or Met;
Xaa at position 12 is Ser. Tyr, or Thr;
Xaa at position 15 is Val or Leu;
Xaa at position 17 is Asp or Glu;
Xaa at position 46 is Ser or Thr;
Xaa at position 66 is Ala or Gly; and
Xaa at position 69 is Thr or Gln;
   Formula I [SEQ ID NO:7]

A more preferred full-length light chain region for the antibodies of the present invention comprises SEQ ID NO:8. The CDRs based on the definition of Kabat are underlined.

| | |
|---|---|
| Asp Ile Gln Met Thr Gln Ser Pro Ser Ser Leu Ser Ala Ser Val | [SEQ ID NO:8] |
| GAC ATG CAG ATG ACC CAG TCT CCA TCT TCC CTG TCT GCA TCT GTA | |
| Gly Asp Arg Val Thr Ile Thr Cys Lys Ala Ser Gln Asp Ile Asp | |
| GGA GAC AGA GTC ACT ATC ACT TGC AAG GCG AGT CAG GAC ATT GAT | |
| Arg Tyr Leu Ser Trp Phe Gln Gln Lys Pro Gly Lys Ala Pro Lys | |
| AGG TAT TTA AGT TGG TTC CAG CAG AAA CCA GGG AAA GCT CCT AAG | |
| Ser Leu Ile Tyr Arg Val Lys Arg Leu Val Asp Gly Val Pro Ser | |
| TCC CTG ATC TAT CGT GTA AAG AGA TTG GTA GAT GGG GTC CCA TCA | |
| Arg Phe Ser Gly Ser Ala Ser Gly Thr Asp Tyr Thr Leu Thr Ile | |
| AGG TTC AGT GGC AGC GCA TCT GGG ACA GAT TAT ACT CTC ACC ATC | |
| Ser Ser Leu Gln Pro Glu Asp Phe Ala Thr Tyr Tyr Cys Leu Gln | |
| AGC AGC CTG CAG CCT GAA GAT TTC GCA ACC TAT TAT TGT CTA CAG | |
| Tyr Asp Glu Phe Pro Tyr Thr Phe Gly Gln Gly Thr Lys Leu Glu | |
| TAT GAT GAG TTT CCG TAC ACG TTC GGA CAG GGG ACC AAG CTG GAA | |
| Ile Lys Arg Thr Val Ala Ala Pro Ser Val Phe Ile Phe Pro Pro | |
| ATA AAA CGA ACT GTG GCT GCA CCA TCT GTC TTC ATC TTC CCG CCA | |
| Ser Asp Glu Gln Leu Lys Ser Gly Thr Ala Ser Val Val Cys Leu | |
| TCT GAT GAG CAG TTG AAA TCT GGA ACT GCC TCT GTT GTG TGC CTG | |
| Leu Asn Asn Phe Tyr Pro Arg Glu Ala Lys Val Gln Trp Lys Val | |
| CTG AAT AAC TTC TAT CCC AGA GAG GCC AAA GTA CAG TGG AAG GTG | |
| Asp Asn Ala Leu Gln Ser Gly Asn Ser Gln Glu Ser Val Thr Glu | |
| GAT AAC GCC CTC CAA TCG GGT AAC TCC CAG GAG AGT GTC ACA GAG | |
| Gln Asp Ser Lys Asp Ser Thr Tyr Ser Leu Ser Ser Thr Leu Thr | |
| CAG GAC AGC AAG GAC AGC ACC TAC AGC CTC AGC AGC ACC CTG ACG | |
| Leu Ser Lys Ala Asp Tyr Glu Lys His Lys Val Tyr Ala Cys Glu | |
| CTG AGC AAA GCA GAC TAC GAG AAA CAC AAA GTC TAC GCC TGC GAA | |
| Val Thr His Gln Gly Leu Ser Ser Pro Val Thr Lys Ser Phe Asn | |
| GTC ACC CAT CAG GGC CTG AGC TCG CCC GTC ACA AAG AGC TTC AAC | |
| Arg Gly Glu Cys | |
| AGG GGA GAG TGT | |

A preferred signal sequence immediately preceding SEQ ID NO:8 or 7, is as follows:

| | |
|---|---|
| Met Asp Met Arg Thr Pro Ala Gln Phe Leu Gly Ile Phe Phe Phe | [SEQ ID NO:9] |
| ATG GAC ATG AGG ACC CCT GCT CAG TTT CTT GGA ATC TTT TTC TTC | |
| Trp Phe Pro Gly Ile Arg Cys | |
| TGG TTT CCA GGT ATC AGA TGT | |

A preferred heavy chain variable region for the antibodies of the present invention comprises Formula II which is SEQ ID NO:10. The CDRs based on the definition of Kabat are underlined.

1 2 3 4 5 6 7 8 9 10 11 12 13 14 15
Xaa Val Gln Leu Val Gln Ser Gly Ala Glu Val Lys Lys Pro Gly
16 17 18 19 20 21 22 23 24 25 26 27 28 29 30
Ala Ser Val Lys Val Ser Cys Lys Xaa Ser Gly Tyr Thr Phe Xaa
31 32 33 34 35 36 37 38 39 40 41 42 43 44 45
Arg Tyr Trp Ile Glu Trp Xaa Arg Gln Ala Pro Gly Xaa Gly Leu
46 47 48 49 50 51 52 53 54 55 56 57 58 59 60
Glu Trp Xaa Gly Glu Ile Leu Pro Xaa Xaa Xaa Asn Ile Asn Tyr
61 62 63 64 65 66 67 68 69 70 71 72 73 74 75
Asn Glu Lys Phe Lys Gly Xaa Xaa Thr Xaa Thr Ala Asp Xaa Ser
76 77 78 79 80 81 82 83 84 85 86 87 88 89 90
Xaa Xaa Thr Ala Tyr Met Glu Leu Ser Ser Leu Xaa Ser Glu Asp
91 92 93 94 95 96 97 98 99 100 101 102 103 104 105
Thr Ala Val Tyr Tyr Cys Ser Thr Ile Tyr Tyr Asp Tyr Asp Gln
106 107 108 109 110 111 112 113 114 115 116 117 118 119 120
Gly Phe Thr Tyr Trp Gly Gln Gly Thr Leu Val Thr Val Ser Ser

Xaa at position 1 is Gln or Glu;
Xaa at position 24 is Val, Ala, or Ser;
Xaa at position 30 is Ser or Thr;
Xaa at position 37 is Val or Ile;
Xaa at position 43 is Lys, Gln, or His;
Xaa at position 48 is Ile or Met;
Xaa at position 54 is Gly, Ala, Ser, Val, Thr, Leu, Ile, Met, Phe, Tyr or Trp;
Xaa at position 55 is Asn, Gln, Arg, Asp, Ser, Gly, or Ala;
Xaa at position 56 is Gly, Ala, Ser, Val, Thr, Leu, Ile, Met, Phe, Tyr or Trp;
Xaa at position 67 is Lys or Arg;
Xaa at position 68 is Ala or Val;
Xaa at position 70 is Ile, Met, or Val;
Xaa at position 74 is Thr or Ser;
Xaa at position 76 is Thr or Ser;
Xaa at position 77 is Asp, Glu, or Ser; and
Xaa at position 87 is Arg or Ser
   Fornula II [SEQ ID NO:10]

A more preferred full-length heavy chain region for the antibodies of the present invention comprises SEQ ID NO: 11. The CDRs based on the definition of Kabat are underlined.

| | |
|---|---|
| Gln Val Gln Leu Val Gln Ser Gly Ala Glu Val Lys Lys Pro Gly | (SEQ ID NO:11) |
| CAG GTT CAG CTG GTG CAG TCT GGA GCT GAG GTG AAG AAG CCT GGG | |
| Ala Ser Val Lys Val Ser Cys Lys Val Ser Gly Tyr Thr Phe Ser | |
| GCC TCA GTG AAG GTG TCC TGC AAG GTG TCT GGC TAC ACA TTC AGT | |
| Arg Tyr Trp Ile Glu Trp Val Arg Gln Ala Pro Gly Lys Gly Leu | |
| AGG TAT TGG ATA GAG TGG GTT AGA CAG GCA CCT GGA AAA GGC CTT | |
| Glu Trp Ile Gly Glu Ile Leu Pro Gly Ser Gly Asn Ile Asn Tyr | |
| GAG TGG ATT GGA GAG ATT TTA CCT GGA TCC GGA AAT ATT AAC TAC | |
| Asn Glu Lys Phe Lys Gly Lys Ala Thr Ile Thr Ala Asp Thr Ser | |
| AAT GAG AAG TTC AAG GGC AAG GCC ACA ATC ACA GCA GAT ACA TCC | |
| Thr Asp Thr Ala Tyr Met Glu Leu Ser Ser Leu Arg Ser Glu Asp | |
| ACA GAT ACA GCC TAC ATG GAA CTC AGC AGC CTG AGG TCT GAG GAC | |
| Thr Ala Val Tyr Tyr Cys Ser Thr Ile Tyr Tyr Asp Tyr Asp Gln | |
| ACA GCC GTC TAT TAT TGT TCA ACA ATC TAC TAT GAT TAC GAC CAG | |
| Gly Phe Thr Tyr Trp Gly Gln Gly Thr Leu Val Thr Val Ser Ser | |
| GGG TTT ACT TAC TGG GGC CAA GGG ACT CTG GTC ACT GTT TCT TCT | |
| Ala Ser Thr Lys Gly Pro Ser Val Phe Pro Leu Ala Pro Ser Ser | |
| GCC TCC ACC AAG GGC CCA TCG GTC TTC CCC CTC GCA CCC TCC TCC | |
| Lys Ser Thr Ser Gly Gly Thr Ala Ala Leu Gly Cys Leu Val Lys | |
| AAG AGC ACC TCT GGG GGC ACA GCG GCC CTG GGC TGC CTG GTC AAG | |
| Asp Tyr Phe Pro Glu Pro Val Thr Val Ser Trp Asn Ser Gly Ala | |
| GAC TAC TTC CCC GAA CCG GTG ACG GTG TCG TGG AAC TCA GGC GCC | |
| Leu Thr Ser Gly Val His Thr Phe Pro Ala Val Leu Gln Ser Ser | |
| CTG ACC AGC GGC GTG CAC ACC TTC CCG GCT GTC CTA CAG TCC TCA | |
| Gly Leu Tyr Ser Leu Ser Ser Val Val Thr Val Pro Ser Ser Ser | |
| GGA CTC TAC TCC CTC AGC AGC GTG GTG ACC GTG CCC TCC AGC AGC | |
| Leu Gly Thr Gln Thr Tyr Ile Cys Asn Val Asn His Lys Pro Ser | |
| TTG GGC ACC CAG ACC TAC ATC TGC AAC GTG AAT CAC AAG CCC AGC | |
| Asn Thr Lys Val Asp Lys Lys Val Glu Pro Lys Ser Cys Asp Lys | |
| AAC ACC AAG GTG GAC AAG AAA GTT GAG CCC AAA TCT TGT GAC AAA | |
| Thr His Thr Cys Pro Pro Cys Pro Ala Pro Glu Leu Leu Gly Gly | |
| ACT CAC ACA TGC CCA CCG TGC CCA GCA CCT GAA CTC CTG GGG GGA | |
| Pro Ser Val Phe Leu Phe Pro Pro Lys Pro Lys Asp Thr Leu Met | |
| CCG TCA GTC TTC CTC TTC CCC CCA AAA CCC AAG GAC ACC CTC ATG | |
| Ile Ser Arg Thr Pro Glu Val Thr Cys Val Val Val Asp Val Ser | |
| ATC TCC CGG ACC CCT GAG GTC ACA TGC GTG GTG GTG GAC GTG AGC | |
| His Glu Asp Pro Glu Val Lys Phe Asn Trp Tyr Val Asp Gly Val | |
| CAC GAA GAC CCT GAG GTC AAG TTC AAC TGG TAC GTG GAC GGC GTG | |
| Glu Val His Asn Ala Lys Thr Lys Pro Arg Glu Glu Gln Tyr Asn | |
| GAG GTG CAT AAT GCC AAG ACA AAG CCG CGG GAG GAG CAG TAC AAC | |
| Ser Thr Tyr Arg Val Val Ser Val Leu Thr Val Leu His Gln Asp | |
| AGC ACG TAC CGT GTG GTC AGC GTC CTC ACC GTC CTG CAC CAG GAC | |
| Trp Leu Asn Gly Lys Glu Tyr Lys Cys Lys Val Ser Asn Lys Ala | |
| TGG CTG AAT GGC AAG GAG TAC AAG TGC AAG GTC TCC AAC AAA GCC | |
| Leu Pro Ala Pro Ile Glu Lys Thr Ile Ser Lys Ala Lys Gly Gln | |
| CTC CCA GCC CCC ATC GAG AAA ACC ATC TCC AAA GCC AAA GGG CAG | |
| Pro Arg Glu Pro Gln Val Tyr Thr Leu Pro Pro Ser Arg Asp Glu | |
| CCC CGA GAA CCA CAG GTG TAC ACC CTG CCC CCA TCC CGG GAT GAG | |
| Leu Thr Lys Asn Gln Val Ser Leu Thr Cys Leu Val Lys Gly Phe | |
| CTG ACC AAG AAC CAG GTC AGC CTG ACC TGC CTG GTC AAA GGC TTC | |
| Tyr Pro Ser Asp Ile Ala Val Glu Trp Glu Ser Asn Gly Gln Pro | |
| TAT CCC AGC GAC ATC GCC GTG GAG TGG GAG AGC AAT GGG CAG CCG | |
| Glu Asn Asn Tyr Lys Thr Thr Pro Pro Val Leu Asp Ser Asp Gly | |
| GAG AAC AAC TAC AAG ACC ACG CCT CCC GTG CTG GAC TCC GAC GGC | |
| Ser Phe Phe Leu Tyr Ser Lys Leu Thr Val Asp Lys Ser Arg Trp | |
| TCC TTC TTC CTC TAC AGC AAG CTC ACC GTG GAC AAG AGC AGG TGG | |
| Gln Gln Gly Asn Val Phe Ser Cys Ser Val Met His Glu Ala Leu | |
| CAG CAG GGG AAC GTC TTC TCA TGC TCC GTG ATG CAT GAG GCT CTG | |
| His Asn His Tyr Thr Gln Lys Ser Leu Ser Leu Ser Pro Gly Lys | |
| CAC AAC CAC TAC ACG CAG AAG AGC CTC TCC CTG TCT CCG GGT AAA | |

A preferred signal sequence immediately preceding SEQ ID NO: 11 or 10, is the following:

| | |
|---|---|
| Met Glu Trp Thr Trp Val Phe Leu Phe Leu Leu Ser Val | [SEQ ID NO:12] |
| ATG GAA TGG ACC TGG GTC TTT CTC TTC CTC CTG TCA GTA | |
| Thr Ala Gly Val His Ser | |
| ACT GCA GGT GTC CAC TCC | |

The analogs referred to herein are analogs of antibody “Hu007”, a humanized version of mouse monoclonal antibody Mu007 having a light chain B sequence corresponding to SEQ ID NO:7 and a heavy chain sequence corresponding to SEQ ID NO:10.

The analogs of the present invention are the result of site-directed mutagenesis at positions 54, 55, and 56 of the heavy chain CDR2 region. The analogs reduce or eliminate deamidation of position 55. The process of deamidation is a well-recognized phenomenon that may impact the stability/activity of proteins and may occur at an Asn or Gln residue. Deamidation at Asn occurs more frequently and the rate of deamidation is highly dependent on the primary, secondary and tertiary structure of the protein. Studies using model peptides indicate that when Asn is followed by a residue with a small side chain, i.e. Gly (the characteristic -NG- sequence motif), the deamidation rate can be 100-fold faster than Asn followed by a more bulky residue such as Val. The heavy chain of antibody Hu007 contains three -NG- sequence motifs (positions 55-56, 318-319, and 387-388). Unexpectedly, the Asn at position 55 is the only major site of deamidation (Example 12). Hu007 analog N55D which mimics the fully deamidated antibody has about 15-20-fold lower potency (Example 10) as measured in an in vitro cell based assay and about 15-fold loss of its binding affinity to IL-1β measured by BIAcore analysis (Example 13).

The present invention encompasses analogs that contain the heavy chain CDR2, SEQ ID NO:1, and one or more of the CDRs of antibody Mu007. The CDRs encompassed by the present invention are the hypervariable regions of the Mu007 antibody, which provide the majority of contact residues for the binding of the antibody to a specific IL-1β epitope. Thus, the CDRs described herein can be used to make full-length antibodies as well as functional fragments or other proteins which when attached to the CDRs maintain the CDRs in an active structural conformation such that the binding affinity of the protein employing the CDRs for mature IL-1β increases compared to the binding affinity of Mu007, is the same as the binding affinity of Mu007, or does not decrease by more than A 10-fold compared to the binding affinity of the Mu007 antibody. Preferably the binding affinity does not decrease by more than 5-fold compared to the binding affinity of the Mu007 antibody. Most preferably the binding affinity is within 3-fold the binding affinity of the Mu007 antibody.

The binding affinity of the Mu007 antibody was determined using surface plasmon resonance (BIAcore™). In these experiments antibody was immobilized at low density on a BIAcore™ chip and ligand was flowed past. Build up of mass at the surface of the chip was measured. This analytical method allows the determination in real time of both on and off rates for binding. The Mu007 antibody has an affinity of approximately 6.2 picomolar (See Example 9). A preferred humanized antibody of the present invention, Hu007 had an affinity of approximately 10.2 picomolar (See Example 9). The Mu007 and Hu007 antibodies bind specifically to IL-1β and not other IL-1 family members or structurally related proteins within the same species (See Example 9).

The binding affinity of the analogs of the present invention was also determined using surface plasmon resonance (BIAcore™) (Example 13). It is also preferred that the analogs of the present invention bind specifically to IL-1β. For example, the most preferred analog of the present invention, heavy chain CDR2, N55S (Asn at position 55 is substituted with Ser), has a binding affinity to IL-1β within 3-fold the binding affinity of the Hu007 antibody (Table 3, Example 13).

The analogs of the present invention reduce or eliminate the deamidation of amino acid 55 in the heavy chain CDR2 region. The preferred analogs have increased stability when compared to wild type (WT) Hu007 antibody (Example 14). For example, analog G56V reduces deamidation compared to WT and analogs N55D, N55S, and N55Q essentially eliminate deamidation at this site compared to WT (Table 4, Example 14).

It is also preferred that the analogs of the present invention neutralize the biological activity of IL-1β. Two different assays were employed to test the ability of Mu007, Hu007, and the analogs of the present invention to neutralize IL-1β activity. A murine cell line which requires low levels of IL-1β for proliferation was used in the first assay. Human IL-1β was present at a constant level in the medium and a dilution series of each antibody was added. Inhibition of proliferation provided a measurement of the efficacy of the antibody's ability to block IL-1β activation of the IL-1 receptor. Proliferation measurements for different concentrations of antibody resulted in an average IC50 value of 220 picomolar for Mu007 and 480 picomolar for Hu007 (See example 10). It is preferred that the analogs of the present invention have an IC50 potency which is better than, the same as, or within 10-fold that of Mu007. Preferably the IC50 potency is within 5-fold that of Mu007. Most preferably the IC50 potency is within 3-fold that of Mu007. “IC50” as referred to herein is the measure of potency of an antibody to inhibit the activity of human IL-1β. IC50 is the concentration of antibody that results in 50% IL-1β inhibition in a single concentration experiment. The IC50 can be measured by any assay that detects inhibition of human IL-1β activity. However, the IC50 values obtained may vary depending on the assay used. There may even be some variability between experiments using the same assay. For example, the condition of the IL-1β dependent cells discussed herein, has an effect on the IC50 values obtained. Thus, the critical value for the purposes of the present invention is a value relative to that obtained using Mu007, Hu007, or analogs of the present invention in a single experiment (Table 3, Example 10).

Neither Hu007 nor the analogs of the present invention cross-react with mouse IL-1β making it difficult to use a mouse model to test neutralizing activity in vivo. However, one consequence of the proinflammatory activity of IL-1β, is the induction of IL-6, another proinflammatory cytokine that mediates some of the non-local effects of IL-1β. Human IL-1β is able to bind and stimulate the mouse IL-1β receptor, leading to an elevation of mouse IL-6. Thus, an antibody with neutralizing activity would block the induction of IL-6 in a mouse given a dose of human IL-1β. Both Mu007 and Hu007 demonstrated potent neutralization of human IL-1β in the murine model of inflammatory stimulation. The humanized antibody was approximately one third as efficacious as the Mu007 antibody (See example 11).

The invention also encompasses analogs wherein the Mu007 CDRs have been grafted into a human framework region or a human framework variant such as in Hu007 and then modified or mutated to enhance binding affinity or other biological properties such as the ability of the antibody to neutralize IL-1β activity at specific concentrations which can be expressed as an IC50 value.

It is preferred that the analogs of the present invention bind the same epitope on human IL-1β as the Mu007 and Hu007 antibodies. In addition, the invention encompasses antibodies that bind epitopes that overlap with or include the epitope bound by the Mu007 and Hu007 antibodies provided those antibodies have the ability to neutralize human IL-1β in vivo.

The present invention encompasses the. discovery of a specific region of the 165 amino acid mature form of human IL-1β wherein the binding of an antibody to that region completely neutralizes activity of the protein. Furthermore, antibodies directed to this specific region of mature IL-1β are specific in that they do not cross react with other IL-1 family members or related proteins. While the invention encompasses any analog that binds this epitope and neutralize IL-1β activity, it is preferred that the analogs employ the heavy chain CRD2, SEQ ID NO:1 and at least one of the CDRs present in Mu007. Antibodies that neutralize IL-1β activity prevent the mature IL-1β protein from binding to its receptor and/or initiating a signal transduction pathway.

The present invention also is directed to recombinant DNA encoding antibodies which, when expressed, specifically bind to the same epitope that Mu007 and Hu007 bind to and have potent in vivo neutralizing activity.

Preferably, the DNA encodes antibodies that, when expressed, comprise SEQ ID NO:1 and one or more of the heavy and light chain Mu007 CDRs [SEQ ID NO:2, 3, 4, 5, and 6]. Exemplary DNA sequences which, on expression, code for the polypeptide chains comprising the heavy and light chain CDRs of the Mu007 and Hu007 antibodies are represented as SEQ ID NO:8 and 11. Due to the degeneracy of the genetic code, other DNA sequences can be readily substituted for the exemplified sequences.

DNA encoding the analogs of the present invention will typically further include an expression control polynucleotide sequence operably linked to the antibody coding sequences, including naturally-associated or heterologous promoter regions. Preferably, the expression control sequences will be eukaryotic promoter systems in vectors capable of transforming or transfecting eukaryotic host cells, but control sequences for prokaryotic hosts may also be used. Once the vector has been incorporated into the appropriate host cell line, the host cell is propagated under conditions suitable for expressing the nucleotide sequences, and, as desired, the collection and purification of the light chains, heavy chains, light/heavy chain dimers or intact antibodies, binding fragments or other immunoglobulin forms.

The nucleic acid sequences of the present invention capable of ultimately expressing the desired analogs can be formed from a variety of different polynucleotides (genomic or cDNA, RNA, synthetic oligonucleotides, etc.) and components (e.g., V, J, D, and C regions), using any of a variety of well known techniques. Joining appropriate genomic and synthetic sequences is a common method of production, but cDNA sequences may also be utilized.

Human constant region DNA sequences can be isolated in accordance with well known procedures from a variety of human cells, but preferably from immortalized B-cells. Suitable source cells for the polynucleotide sequences and host cells for immunoglobulin expression and secretion can be obtained from a number of sources well known in the art.

As described herein, in addition to the analogs specifically described herein, other “substantially homologous” modified analogs can be readily designed and manufactured utilizing various recombinant DNA techniques well known to those skilled in the art. For example, the framework regions can vary from the native sequences at the primary structure level by several amino acid substitutions, terminal and intermediate additions and deletions, and the like. Moreover, a variety of different human framework regions may be used singly or incombination as a basis for the humanized immunoglobulins of the present invention. In general, modifications of the genes may be readily accomplished by a variety of well-known techniques, such as site-directed mutagenesis.

Alternatively, polypeptide fragments comprising only a portion of the primary antibody structure may be produced, which fragments possess one or more immunoglobulin activities (e.g., complement fixation activity). These polypeptide fragments may be produced by proteolytic cleavage of intact antibodies by methods well known in the art, or by inserting stop codons at the desired locations in vectors using site-directed mutagenesis, such as after CH1 to produce Fab fragments or after the hinge region to produce F(ab′)₂ fragments. Single chain antibodies may be produced by joining VL and VH with a DNA linker.

As stated previously, the polynucleotides will be expressed in hosts after the sequences have been operably linked to (i.e., positioned to ensure the functioning of) an expression control sequence. These expression vectors are typically replicable in the host organisms either as episomes or as an integral part of the host chromosomal DNA. Commonly, expression vectors will contain selection markers, e.g., tetracycline, neomycin, and dihydrofolate reductase, to permit detection of those cells transformed with the desired DNA sequences.

E. coli is a prokaryotic host useful particularly for cloning the polynucleotides of the present invention. Other microbial hosts suitable for use include bacilli, such as Bacillus subtilus, and other enterobacteriaceae, such as Salmonella, Serratia, and various Pseudomonas species. In these prokaryotic hosts, one can also make expression vectors, which will typically contain expression control sequences compatible with the host cell (e.g., an origin of replication). In addition, any of a number of well-known promoters may be present, such as the lactose promoter system, a tryptophan (trp) promoter system, a beta-lactamase promoter system, or a promoter system from phage lambda. The promoters will typically control expression, optionally with an operator sequence, and have ribosome binding site sequences and the like, for initiating and completing transcription and translation.

Other microbes, such as yeast, may also be used for expression. Saccharomyces is a preferred host, with suitable vectors having expression control sequences, such as promoters, including 3-phosphoglycerate kinase or other glycolytic enzymes, and an origin of replication, termination sequences and the like as desired.

In addition to microorganisms, plant cells can also be modified to create transgenic plants that express the antibody or antigen binding portion of the invention. Optimal methods of plant transformation vary depending on the type of plant (see WO00/53794, U.S. Pat. Nos. 5,202,422 and 6,096,547 and Giddings et al., Nature Biotechnology 18:1151 (2000)).

Mammalian tissue cell culture may also be used to express and produce the polypeptides of the present invention. Eukaryotic cells are actually preferred, because a number of suitable host cell lines capable of secreting intact immunoglobulins have been developed in the art, and include the CHO cell lines, various COS cell lines, Syrian Hamster Ovary cell lines, HeLa cells, myeloma cell lines, transformed B-cells, human embryonic kidney cell lines, or hybridomas. Preferred cell lines are CHO and myeloma cell lines such as SP2/0 and NS0.

Expression vectors for these cells can include expression control sequences, such as an origin of replication, a promoter, an enhancer, and necessary processing information sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma. Virus, cytomegalovirus and the like. Preferred polyadenylation sites include sequences derived from SV40 and bovine growth hormone.

The vectors containing the polynucleotide sequences of interest (e.g., the heavy and light chain encoding sequences and expression control sequences) can be transferred into the host cell by well-known methods, which vary depending on the type of cellular host. For example, calcium chloride transfection is commonly utilized for prokaryotic cells, whereas calcium phosphate treatment or electroporation may be used for other cellular hosts.

In another embodiment, antibodies or antigen-binding portions thereof of the invention can be expressed in an animal (e.g., a mouse) that is transgenic for human immunoglobulin genes (see e.g., Taylor, L. D. et al. Nucl. Acids Res., 20:6287-6295(1992)). Transgenic animals that are capable, upon immunization, of producing a full repertoire of human antibodies in the absence of endogenous immunoglobulin production can be employed. Transfer of the human germn-line immunoglobulin gene array in such germ-line mutant mice will result in the production of human antibodies upon antigen challenge (see, e.g., Jakobovits et al., Proc. Natl. Acad. Sci. USA, 90:2551 -2555, (1993); Jakobovits et al., Nature, 362:255-258, (1993); Bruggemann et al., Nature Biotechnology 14:826 (1996); Gross, J. A., et al., Nature, 404:995-999 (2000); and U.S. Pat. Nos. 5,874,299, 5,814,318, and 5,789,650). Human antibodies can also be produced in phage display libraries (Hoogenboom and Winter, J. Mol. Biol., 227:381 (1992); Marks et al., J. Mol. Biol., 222:581 (1991)). The techniques of Cole et al. and Boemer et al. are also available for the preparation of human monoclonal antibodies (Cole et al., Monoclonal Antibodies and Cancer Therap, Alan R. Liss, p. 77 (1985) and Boerner et al., J. Immunol., 147(1):86-95 (1991)). In addition, human monoclonal antibodies can be produced in mammal's milk through the creation of transgenic animals that selectively express foreign antibody genes in mammary epithelial cells (U.S. Pat. No. 5,849,992).

Once expressed, the analogs can be purified according to standard procedures, including ammonium sulfate precipitation, ion exchange, affinity (e.g. Protein A), reverse phase, hydrophobic interaction column chromatography, gel electrophoresis, and the like. Substantially pure immunoglobulins having at least about 90 to 95% purity are preferred, and 98 to 99% or more purity most preferred, for pharmaceutical uses. Once purified, partially or to homogeneity as desired, the polypeptides may then be used therapeutically or prophylactically, as directed herein.

This invention also relates to a method of treating humans experiencing an IL-1β mediated inflammatory disorder, which comprises administering an effective dose of an anti-IL-1β analog to a patient in need thereof. The analogs of the present invention bind to and prevent IL-1β from binding an IL-1β receptor and initiating a signal. Various IL-1β-mediated disorders include rheumatoid arthritis (RA), osteoarthritis (OA), allergy, septic or endotoxic shock, septicemia, stroke, asthma, graft versus host disease, Crohn's disease, and other inflammatory bowel diseases. Preferably, the anti-IL-1β analogs encompassed by the present invention are used to treat RA and/or OA.

Patients with RA suffer from chronic swelling and inflammation of the joints and ongoing destruction of cartilage and bone. IL-1β and TNF-α are the most critical cytokines in the pathogenesis of RA. However, while both IL-1β and TNF-α mediate inflammation, IL-1β is the primary mediator of bone and cartilage destruction. Activated monocytes and fibroblasts in the synovial tissue produce IL-1β which in turn stimulates the production of additional pro-inflammatory cytokines, prostaglandins, and matrix metalloproteases. The synovial lining becomes hypertrophied, invading and eroding bone and cartilage.

Disease-modifying antirheumatic drugs (DMARDS) such as hydroxychloroquine, oral or injectable gold, methotrexate, azathioprine, penicillamine, and sulfasalazine have been used with modest success in the treatment of RA. Their activity in modifying the course of RA is believed to be due to suppression or modification of inflammatory mediators such as IL-1β. Methotrexate, for example, at doses of 7.5 to 10 mg per week caused a reduction in IL-1β plasma concentrations in RA patients. Similar results have been seen with corticosteroids. Thus, the anti-IL-1β analogs of the present invention may be used alone or in combinations with DMARDS, which may act to reduce IL-1β protein levels in plasma.

An effective amount of the anti-IL-1β analogs of the present invention is that amount which provides clinical efficacy without intolerable side effects or toxicity. Clinical efficacy for RA patients can be assessed using the American College of Rheumatology Definition of Improvement (ACR20). A patient is considered a responder if they show a 20% improvement in the tender joint count, swollen joint count, and 3 of 5 other components which include patient pain assessment, patient global assessment, physician global assessment, Health Assessment Questionnaire, and serum C-reactive protein. Prevention of structural damage can be assessed by the van der Heijde modification of the Sharp Scoring system for radiographs (erosion count, joint space narrowing).

The anti-IL-1β analogs of the present invention can also be used to treat patients suffering from OA. OA is the most common disease of human joints and is characterized by articular cartilage loss and osteophyte formation. Clinical features include joint pain, stiffness, enlargement, instability, limitation of motion, and functional impairment. OA has been classified as idiopathic (primary) and secondary forms. Criteria for classification of OA of the knee and hip have been developed by the American College of Rheumatology on the basis of clinical, radiographic, and laboratory parameters.

The anti-IL-1β analogs of the present invention can also be used for the manufacture of a medicament to treat a subject with RA or OA. Additionally, the anti-IL-1β analogs of the present invention can be used for the manufacture of a medicament to inhibit cartilage destruction in a subject in need thereof.

An effective amount of the anti-IL-1β analogs of the present invention is the amount which shows clinical efficacy in OA patients as measured by the improvement in pain and function as well as the prevention of structural damage. Improvements in pain and function can be assessed using the pain and physical function subscales of the WOMAC OA Index. The index probes clinically important patient-relevant symptoms in the areas of pain, stiffness, and physical function. Measuring joint space width on radiographs of the knee or hip can assess prevention of structural damage.

The analogs of the present invention are administered using standard administration techniques, preferably peripherally (i.e. not by administration into the central nervous system) by intravenous, intraperitoneal, subcutaneous, pulmonary, transdermal, intramuscular, intranasal, buccal, sublingual, oral, or suppository administration.

The pharmaceutical compositions for administration are designed to be appropriate for the selected mode of administration, and pharmaceutically acceptable excipients such as, buffers, surfactants, preservatives, solubilizing agents, isotonicity agents, stabilizing agents and the like are used as appropriate. Reminpton's Pharmaceutical Sciences, Mack Publishing Co., Easton Pa., latest edition, incorporated herein by reference, provides a compendium of formulation techniques as are generally known to practitioners.

The concentration of the anti-IL-1β analog in formulations may be from as low as about 0.1% to as much as 15 or 20% by weight and will be selected primarily based on fluid volumes, viscosities, stability, and so forth, in accordance with the particular mode of administration selected. Preferred concentrations of the IL-1β antibody will generally be in the range of 1 to about 100 mg/mL, preferably, 10 to about 50 mg/mL.

The formulation may include a buffer. Preferably the buffer is a citrate buffer or a phosphate buffer or a combination thereof. Generally, the pH of the formulation is between about 4 and about 8. Preferably, the pH is between about 5 and about 7.5. More preferably, the pH is between about 5.5 and about 7. The pH of the formulation can be selected to balance analog stability (chemical and physical) and comfort to the patient when administered. The formulation may also include a salt such as NaCl. In addition, the formulation may include a detergent to prevent aggregation and aid in maintaining stability. For example, pluronic detergents, Tween (80 or 20), or a combination of pluronics and Tween were shown to be compatible with the Hu007 antibody.

The formulation may be sterile filtered after making the formulation, or otherwise made microbiologically acceptable. A preservative such as m-cresol or phenol, or a mixture thereof may be added to prevent microbial growth and contamination.

A typical composition for intravenous infusion could have a volume as much as 250 mL of fluid, such as sterile Ringer's solution, and 1-100 mg per mL, or more in antibody concentration. Therapeutic agents of the invention can be a stable solution, a frozen plug, or a lyophilized plug for storage and reconstituted in a suitable sterile carrier prior to use. Lyophilization and reconstitution can lead to varying degrees of antibody activity loss (e.g. with conventional immunoglobulins, IgM antibodies tend to have greater activity loss than IgG antibodies). Dosages may have to be adjusted to compensate.

Although the foregoing methods appear the most convenient and most appropriate for administration of proteins such as humanized antibodies, by suitable adaptation, other techniques for administration, such as transdermal administration and oral administration may be employed provided proper formulation is designed. In addition, it may be desirable to employ controlled release formulations using biodegradable films and matrices, or osmotic mini-pumps, or delivery systems based on dextran beads, alginate, or collagen. In summary, formulations are available for administering the analogs of the invention and may be chosen from a variety of options.

Typical dosage levels can be optimized using standard clinical techniques and will be dependent on the mode of administration and the condition of the patient. Generally, doses will be in the range of 10 μg/kg/month to 40 mg/kg/month.

The invention is illustrated by the following examples that are not intended to be limiting in any way.

### EXAMPLE 1

Mu007 Variable Regions:

The Mu007 light and heavy chain variable region cDNAs were cloned from a hybridoma cell line. Several light and heavy chain clones were sequenced from two independent PCR reactions. The functional light chain variable sequence was typical of a functional mouse kappa chain variable region and was found to belong to subgroup V based on the definition of Kabat (Johnson, G. and Wu, T. T. (2000) Nucleic Acids Res. 28: 214-218). For the heavy chain, a unique sequence homologous to a typical mouse heavy chain variable region was identified. Mu007 variable heavy chain was classified to subgroup II(A) based on the definition of Kabat (Johnson and Wu, 2000). The cDNA sequences coding light and heavy chain variable regions are represented as SEQ ID NO: 1 and 2, respectively.

### EXAMPLE 2

Hu007 Variable Regions:

The human variable region framework used as an acceptor for Mu007 CDRs was constructed and amplified using eight overlapping synthetic oligonucleotides ranging in length from approximately 65 to 80 bases (He, et al. (1998) J. Immunol. 160: 1029-1035). The oligonucleotides were annealed pairwise and extended with the Klenow fragment of DNA polymerase I, yielding four double-stranded fragrnents. The resulting fragments were denatured, annealed pairwise, and extended with Klenow, yielding two fragments. These fragments were denatured, annealed pairwise, and extended once again, yielding a full-length gene. The PCR-amplified fragments were gel-purified and cloned into pCR4Blunt vector. After sequence confirmation, the variable light and variable heavy genes were digested with MluI and XbaI, gel-purified, and subcloned respectively into vectors for expression of light and heavy chains to make pVk-Hu007 and pVgl-Hu007.

### EXAMPLE 3

Cloning and Expression of Hu007 and Hu007 Analogs

Hu007:

Mouse myeloma cell line Sp2/0-Ag14 (hereinafter, Sp2/0) was obtained from the ATCC and maintained in DME medium containing 10% FBS (Cat # SH30071.03, Hyclone, Logan, Utah) at 37° C.

Stable transfection into the mouse myeloma cell line Sp2/0 was accomplished by electroporation using a Gene Pulser apparatus (BioRad, Hercules, Calif.) at 360V and 25 μF according to the manufacturer's instructions. Before transfection, pVk-Hu007 and pVgl-Hu007 plasmid DNAs were linearized using Fspl. Approximately 1 Sp2/0 cells were transfected with 30 μg of pVk-Hu007 and 60 μg of pVgl-Hu007. The transfected cells were suspended in DME medium containing 10% FBS and plated into several 96-well plates. After 48 hr, cells were selected for gpt expression using selection media (DME medium containing 10% FBS, HT media supplement, 0.3 mg/mL xanthine and 1 μg/mL mycophenolic acid). Approximately 10 days after the initiation of selection, culture supernatants were assayed for antibody production by ELISA (See Example 7). High-yielding clones were expanded in DME medium containing 10% FBS and further analyzed for antibody expression. Selected clones were then adapted to growth in serum free medium (Hybridoma SFM, Cat. # 12045-076, Life Technologies, Rockville, Md.). This was accomplished by splitting the cells gradually in Hybridoma SFM, usually by a 25 to 50% split each time, until the serum level was below 0.1%. Thereafter, the transfectant was maintained in Hybridoma SFM. The cell density was maintained between 2×10⁵/mL and 10⁶/mL.

CHO-DG44 cells were transfected with 50 μg of pVk-Hu007 and 50 μg of pVgl-Hu007 (genomic transfection) or 50 μg of an expression vector containing cDNA corresponding to the Hu007 light chain and 50 μg of a vector containing cDNA corresponding to the Hu007 analog heavy chain. Approximately 10⁷ cells were electroporated at 350V/50 μF and 380V/50 μF for the genomic transfection and 350V/71 μF and 380V/71 μF for the cDNA transfection. Cells were incubated at room temperature and then diluted with 20 mL Growth Medium (ExCell 302 medium+4 mM L-Glutamine+1× hypoxanthine/thymidine reagent+100 ,g/mL dextran sulfate) and allowed to recover for 72 hours in a 37° C./5% CO₂ incubator. Cells were selected with medium containing 50 nM methotrexate for the genomic transfectants and 20 nM methotrexate and 200 μg/mL G418 for the cDNA transfectants.

Hu007 Analog Cloning Using Site-Directed Mutagenesis:

Mutagenesis was performed on the CDR2 region of Hu007 using the following procedure: The CDR2 region of Hu007 is defined as (EILPGNGNINYNEKFKG). N55 was mutated to D, Q, and S, and G56 to A and V. A pCID-Hu007HC-cDNA plasmid containing an Ssp I site upstream from the CMV promoter and Ssp I site downstream from the CDR2 region of Hu007 was used as the template to PCR amplify and mutate the CDR2 region of Hu007. The oligonucleotide primers for each mutation are as follows: N55D-(5′ TTCCTTTTTCAATATTATTGAAGCATTTATCAGG 3′) forward primer containing the SspI site in bold and (5′ CATTGTAGTTAATATTTCCATCCCAGGTAAAA 3′) reverse primer containing the Ssp I site in bold and the N55D mutation underlined.

N55Q—The forward primer from N55D was used and the reverse primer as (5′ CATTGTAGTTAATATTTCCTTGTCCAGGTAAAATCTCTC 3′) containing the Ssp I site in bold and N55Q mutation underlined.

N55S—The forward primer from N55D was used and the reverse primer as (5′ CATTGTAGTTAATATTTCCGGATCCAGGTAAAATCTCTC 3′) containing the Ssp I site in bold, the N55S mutation underlined, and introduced a Bam HI site (also in bold) used as a diagnostic cut site.

G56V—The forward primer from N55D was used and the reverse primer as (5′ CATTGTAGTTAATATTTACATTTCCAGGTAAAATCTC 3′) containing the Ssp I site in bold and the G56V mutation underlined.

G56A—The forward primer from N55D was used and the reverse primer as (5′ CATTGTAGTTAATATTTGCATTTCCAGGTAAAATCTC 3′) containing the Ssp I site in bold and the G56A mutation underlined.

The resultant 1316 bp PCR generated fragment was added to TOPO vector (pCR 2.1) and subsequently cleaved with Ssp I, gel purified, and ligated to the vector pCID-Hu007HC-cDNA plasmid that had been previously digested with Ssp I to create mutant vectors (FIG. 4). Each analog vector was used in a co-transfection with the WT Hu007 Light Chain gene, cloned into pcID. The expression work is supported using transient transfection methods with HEK 293-EBNA cells analogous to the procedure described above for CHO cells.

### EXAMPLE 4

Expression and Purification of Hu007 Analogs.

Culture supernatant containing Hu007 analog IgG1 monoclonal antibody was purified by protein-A Sepharose chromatography. Culture supernatant was harvested and 15 loaded onto a protein-A Sepharose column. The column was washed with PBS before the antibody was eluted with 0.1 M glycine-HCl (pH 3.5). After neutralization with 1 M Tris HCl (pH 8), the eluted protein was dialyzed against 3 changes of 2 liters PBS and filtered through a 0.2 μm filter prior to storage at 4° C. Antibody concentration was determined by measuring absorbance at 280 nm (1 mg/mL=1.452 A₂₈₀).

### EXAMPLE 5

Expression and Purification of Mu007:

Hybridoma cells producing Mu007 were first grown in RPMI-1640 medium containing 10% FBS (HyClone), 10 mM HEPES, 2 mM glutamine, 0.1 mM non-essential amino acids, 1 mM sodium pyruvate, 25 μg/mL gentamicin, and then expanded in serum-free media (Hybridoma SFM, Cat # 12045-076, Life Technologies, Rockville, Md.) containing 2% low Ig FBS (Cat # 30151.03, HyClone) to a I liter volume in roller bottles. Mu007 was purified from the culture supernatant by affinity chromatography using a protein-G Sepharose column. Biotinylated Mu007 was prepared using EZ-Link Sulfo-NHS-LC-LC-Biotin (Cat # 21338ZZ, Pierce, Rockford, Ill.).

### EXAMPLE 6

SDS-PAGE Analysis of Isolated Mu007, Hu007, and Hu007 Analog:

SDS-PAGE in Tris-glycine buffer was performed according to standard procedures on a 4-20% gradient gel (Cat # EC6025, Novex, San Diego, Calif.). SDS-PAGE analysis of Mu007, Hu007, and Hu007 analog under non-reducing conditions indicated that both antibodies have a molecular weight of about 150-160 kD. Analysis under reducing conditions indicated that both antibodies were comprised of a heavy chain with a molecular weight of about 50 kD and a light chain with a molecular weight of about 25 kD. The purity of Hu007 analog appeared to be more than 95%.

### EXAMPLE 7

Quantification of Antibody Expression by ELISA:

Wells of 96-well ELISA plates (Nunc-Immuno plate, Cat # 439454, NalgeNunc, Naperville, Ill.) were coated with 100 μL of 1 μg/mL goat anti-human IgG, Fcγ fragment specific, polyclonal antibodies (Cat # 109-005-098, Jackson ImmunoResearch, West Grove, Pa.) in 0.2 M sodium carbonate-bicarbonate buffer (pH 9.4) overnight at 4° C. After washing with Washing Buffer (PBS containing 0.1% Tween 20), wells were blocked with 400 μL of Superblock Blocking Buffer (Cat # 37535, Pierce) for 30 min and then washed with Washing Buffer. Samples containing Hu007 and Hu007 analogs were appropriately diluted in ELISA Buffer (PBS containing 1% BSA and 0.1% Tween 20) and applied to ELISA plates (100 μL per well). As a standard, humanized anti-CD33 IgG1 monoclonal antibody HuM195 (Co et al. (1992) J. Immunol. 148: 1149-1154) was used. ELISA plates were incubated for 2 hr at 37° C. and the wells were washed with Wash Buffer. Then, 100 μL of 1/1,000-diluted HRP-conjugated goat anti-human kappa polyclonal antibodies (Cat # 1050-05, Southern Biotechnology, Birmingham, Ala.) in ELISA Buffer was applied to each well. After incubating for 1 hr at 37° C. and washing with Wash Buffer, 100 μL of ABTS substrate (Cat #s 507602 and 506502, Kirkegaard and Perry Laboratories, Gaithersburg, Md.) was added to each well. Color development was stopped by adding 100 μL of 2% oxalic acid per well. Absorbance was read at 415 nm using an OPTImax microplate reader (Molecular Devices, Menlo Park, Calif.).

### EXAMPLE 8

ELISA Competition:

Wells of 96-well ELISA plates (Nunc-lmmuno plate, Cat # 439454, NalgeNunc) were coated with 100 μL of 0.5. μg/mL of human IL-1β in 0.2M sodium carbonate-bicarbonate buffer (pH 9.4) overnight at 4° C., washed with Wash Buffer, blocked with Superblock blocking buffer for 30 min at 37° C., and washed again with Wash Buffer. A mixture of biotinylated Mu007 (0.16 μg/mL final concentration) and competitor antibody (Mu007, Hu007, or Hu007 analogs starting at 100 μg/mL final concentration and serial 3-fold dilutions) in ELISA Buffer were added in triplicate in a final volume of 100 μL per well. As a no-competitor control, 100 μL of 0.16 μg/mL biotinylated Mu007 was used. As a background control, 100 gLL of ELISA Buffer was used. ELISA plates were incubated at 37° C. for 2 hr. After washing the wells with Washing Buffer, 100 μL of 1 μg/mL HRP-conjugated streptavidin (Jackson ImmunoResearch) was added to each well. ELISA plates were incubated at room temperature for 30 min and washed with Washing Buffer. For color development, 100 μL/well of ABTS substrate was added. Color development was stopped by adding 100 μL/well of 2% oxalic acid. Absorbance was read at 415 nm.

Mu007, Hu007 and Hu007 analogs competed with biotinylated Mu007 in a concentration-dependent manner. The IC₅₀ values of Mu007 and Hu007 in three independent ELISA competition experiments, obtained using the computer software Prism (GraphPad Software Inc., San Diego, Calif.) are shown in Table 1. The relative binding of Hu007 was on average 89% of Mu007.

TABLE 1
Summary of ELISA competition experiments
IC₅₀ (μg/mL)

| | | | | | Std. |
|---|---|---|---|---|---|
| Competitor | Exp. A | Exp. B | Exp. C | Average | Dev. |
| Mu007 | 0.40 | 0.40 | 0.39 | 0.40 | 0.0069 |
| Hu007 | 0.39 | 0.35 | 0.32 | 0.35 | 0.035 |
| Hu007/Mu007 × 100 | 98% | 88% | 82% | 89% | |

### EXAMPLE 9

Binding Affinity and Specificity:

Affinities and specificities of both Hu007 and Mu007 were determined using BlAcore measurements. BIAcore™ is an automated biosensor system that measures molecular interactions. (Karlsson, et al. (1991) J. Immunol. Methods 145: 229-240). In these experiments antibody was immobilized at low density on a BIAcore™ chip. Ethyl-dimethylaminopropyl-carbodiimide (EDC) was used to couple reactive amino groups to purified goat anti-human IgG or goat anti-rabbit IgG to a flow cell of a carboxy-methyl (CM5) BIAcore™ sensor chip. Goat IgG was diluted in sodium acetate buffer, pH 4.0, and immobilized on a flow cell of a CM5 chip using EDC to yield 1000 response units. Unreacted sites were blocked with ehanolamine. A flow rate of 60 μL/min was used. Multiple binding/elution cycles were performed by injection a 100 μL solution of 15 μg/mL Mu007 or Hu007 followed by human IL-1β, mouse IL-1β, rat IL-1β, cynomolgus monkey IL-1β, porcine IL-1β, human IL-1 receptor antagonist, and human IL-1α at decreasing concentrations for each cycle (e.g., 1500, 750, 375, 188, 94, 47, 23.5, 12, and 0 picomolar). Elution was performed with glycine-HCl, pH 1.5. BIAevaluation™ was used to analyze the kinetic data. Table 2 depicts the affinities of Hu007 and Mu007 for human and cynomolgus IL-1β. Mouse IL-1β, rat IL-1β, IL-1 receptor antagonist, and human IL-1α did not bind to Hu007. Cynomolgus and porcine IL-1β had 100% binding to Hu007 relative to human IL-1β.

TABLE 2
Affinities of Hu007 and Mu007 for IL-1β

| | | KD |
|---|---|---|
| Antibody | Target Molecule | (Picomolar) |
| Mu007 | Human IL-1β | 6.2 |
| Hu007 | Human IL-1β | 10.2 |
| Mu007 | Cynomolgus IL-1β | 7.3 |
| Hu007 | Cynomolgus IL-1β | 10.4 |

### EXAMPLE 10

Antibody Potency:

A murine cell requiring low levels of IL-1β for proliferation was used to determine the ability of Hu007 and Mu007 to neutralize human IL-1β. T1165.17 cells which are no longer in log phase growth were washed 3 times with RPMI 1640 (GibcoBRL Cat. # 22400-089) supplemented with 10% fetal calf serum (GibcoBRL Cat. # 10082-147), 1 mM sodium pyruvate, 50 μM beta mercaptoethanol, and an antibiotic/antimycotic (GibcoBRL Cat. # 15240-062). Cells were plated at 5,000 cells per well of a 96 well plate. Human IL-1β was present at a constant level of 0.3 pM and a dilution series of antibody was added. Diluted samples were added and cells were incubated for 20 hours in a 37° C./5% CO₂ incubator at which point 1 μCi ³H-thymidine was added per well and plates incubated an additional 4 hours in the incubator. Cells were harvested and incorporated radioactivity determined by a scintillation counter. FIG. 5 illustrates inhibition of IL-1β stimulated proliferation by Mu007 and Hu007. Average IC50 values calculated from three separate experiments for Mu007 and Hu007 were 220 pM and 480 pM respectively. Additionally, various Hu007 analogs were also assayed for their ability to neutralize human IL-1β stimulated proliferation, Table 3.

TABLE 3
IC₅₀ of Hu007 and Hu007 analogs

| | IC₅₀ |
|---|---|
| Antibody | (Picomolar) |
| Hu007 | 57 ± 1 |
| N55D | 1099 ± 53 |
| N55S | 184 ± 24 |
| N55Q | 2502 ± 140 |
| G56A | 184 ± 7 |
| G56V | 395 ± 24 |

### EXAMPLE 11

Neutralization of Human IL-1β In Vivo:

Human IL-1β is able to bind and stimulate the mouse IL-1β receptor, leading to an elevation of mouse IL-6. Time and dose ranging experiments were undertaken to identify the optimal dose of human IL-1β and the optimal time for induction of mouse IL-6. These experiments indicated that a 3 μg/kg dose of human IL-1β and a time of 2 hours post IL-1β administration gave maximal levels of IL-6 in mouse serum. Mu007 and Hu007 were administered IV to mice one hour prior to an IP injection of human IL-1β. At two hours post IL-1β administration, mice were sacrificed, and IL-6 levels were determined by ELISA. Isotype matched antibodies were used as negative controls. Both Mu007 and Hu007 to inhibit human IL-1β induction of mouse IL-6 in a does dependent manner.

### EXAMPLE 12

Deamidation of Hu007

Deamidation was first monitored by cation exchange chromatogram and IEF gel analysis. Peptide mapping and mass spectrometry analysis were then used to identify and confirm deamidation at Asn55 of CDR2 region of the heavy chain ( . . . EILPGNGNINYNEKFKG . . . ). Effect of pH and temperature on deamidation was further investigated under various solvent conditions. The initial sample around 1.6 mg/mL was stored in PBS, pH 7.4 under refrigerated temperature. This sample is diluted at least 10-fold using various buffers and subsequently concentrated using a Millipore filtration unit with 10,000 MWCO (Millipore Corporation, Bedford, Mass.) to exchange the solvent to conditions listed in Table 4. The extent of deamidation is measured using cation exchange chromatography. The Hu007 samples were run on a Dionex Propac WCX-10 column with a flow rate of 1 mL/min using a linear gradient from 0 to 30% of 10 mM sodium phosphate, 250 mM NaCl, pH 6.5. At least six discrete peaks were observed with cation exchange chromatography. The main species (peak 3) corresponds to Hu007 lacking the C-terminal lysine residue from both heavy chains. After a 7-day incubation at 37° C., increasing amounts of a more acidic form of Hu007 were observed as assessed by cation exchange chromatography and IEF and the relative peak area for the main species (peak 3) decreased. Concomitantly, a significant increase in the relative peak area for peak 1 and 2 was observed. This conversion is highly pH dependent, base catalyzed.

As deamidation occurs, Asn55 of CDR2 region of the heavy chain was converted to either Asp55 or Isoasp55. The area for peak 3 decreases, therefore, percentage of peak area for peak 3 relative to the total peak areas after seven days of incubation in PBS, pH 7.4, at 4° C. and 37° C. was used to monitor the level of deamidation under different conditions. Antibody samples are relatively stable at 4° C. for at least 7 days under these buffer conditions. However, deamidation occurs at 37° C and increases with the increase of pH with minimal effect at pH 6, Table 4.

TABLE 4
Effect of buffer pH on the rate of deamidation.

| | Percent Peak 3 | Percent Peak 3 |
|---|---|---|
| Sample description | (7 days at 37° C.) | (Initial) |
| 0.85 mg/mL, PBS, pH 6, 0.01% | 45 | 44 |
| Tween-80 | | |
| 16.6 mg/mL, PBS, pH 6 | 48 | 44 |
| 11.7 mg/mL, PBS, pH 6.8, 0.005% | 38 | 44 |
| Tween-80 | | |
| 1 mg/mL, PBS, pH 7.4, 0.01% | 28 | 44 |
| pluronic F68 | | |
| 1 mg/mL, PBS, pH 7.4 | 28 | 44 |
| 24 mg/mL, PBS, pH7.4, 0.01% | 31 | 44 |
| Tween-80 | | |
| 1 mg/mL in 10 mM Tris, 150 mM | 37 | 44 |
| NaCl, pH 7.4 | | |

### EXAMPLE 13

Binding Affinity and Specificity of Hu007 Analogs:

Affinities and specificities of several Hu007 analogs were determined using BIAcore measurements. BIAcore™ is an automated biosensor system that measures molecular interactions. (Karlsson, et al. (1991) J. Immunol. Methods 145: 229-240). In these experiments antibody was immobilized at low density on a BIAcore™ chip. Ethyl-dimethylaminopropyl-carbodiimide (EDC) was used to couple reactive amino groups to protein A to a flow cell of a carboxy-methyl (CM5) BIAcore™ sensor chip. Protein A was diluted in sodium acetate buffer, pH 4.5, and immobilized on a flow cell of a CM5 chip using EDC to yield approximately 1000 response units. Unreacted sites were blocked with ethanolamine. A flow rate of 60 μl/min was used. Multiple binding/elution cycles were performed by injection of 10 μl of a 5 μg/mL Hu007 solution, and heavy chain CDR2 analogs N55D, N55S, N55Q, G56A, and G56V, followed by human IL-1β at decreasing concentrations for each cycle (e.g. 1500, 750, 375, 188, and 0 μM). Elution was performed with glycine-HCl, pH 1.5. BIAevaluation™ was used to analyze the kinetic data. Table 5 depicts the affinities of Hu007 and the various Hu007 analogs for human IL-1β.

TABLE 5
Affinities of Hu007 and analogs for IL-1β

| | | KD |
|---|---|---|
| Antibody | Target Molecule | (Picomolar) |
| Hu007 | Human IL-1β | 10.5 |
| N55D | Human IL-1β | 149 |
| N55S | Human IL-1β | 28.1 |
| N55Q | Human IL-1β | 257.0 |
| G56A | Human IL-1β | 33.6 |
| G56V | Human IL-1β | 73.2 |

### EXAMPLE 14

Stability Analysis

Analogs of the present invention were analyzed for the effect of temperature on the rate of Hu007 deamidation monitored by cation exchange chromatography. The Hu007 WT and the several heavy chain CDR2 analogs were incubated at 25° C. or 37° C. for 14 days. Samples were run on a Dionex Propac WXC-10 column with a flow rate of 1 mL/min using a linear gradient from 0 to 30% of 10 mM sodium phosphate, 250mM NaCl, pH 6.5. Aliquots from the stability samples were buffer exchanged into 10 mM phosphate, pH 6.5 prior to loading using a Millipore filtration unit. Significant peaks were identified on the chromatogram as either containing fully intact antibody or analog, deamidated forms or fragments thereof. Stability was determined as a fraction of intact antibody after time of incubation. The fraction of intact antibody was calculated using the peak area for intact antibody divided by the total peak area of intact and deamidated antibody, Table 6. Hu007 analogs G56A and G56V reduced the amount of deamidation over time while analogs at the 55 position prevented deamidation from occurring.

TABLE 6

| | Fraction Intact at 25° C. | | | Fraction Intact at 37° C. | | | | | |
|---|---|---|---|---|---|---|---|---|---|
| Time (days) | WT | G56A | G56V | WT | G56A | G56V | N55D | N55S | N55Q |
| 0 | 0.84 | 0.87 | 0.91 | 0.84 | 0.87 | 0.91 | >0.9 | >0.9 | >0.9 |
| 3 | 0.80 | 0.85 | 0.90 | 0.65 | 0.79 | 0.86 | N.D. | N.D. | N.D. |
| 7 | 0.72 | 0.81 | 0.88 | 0.37 | 0.36 | 0.68 | >0.9 | >0.9 | >0.9 |
| 14 | 0.68 | 0.80 | 0.85 | 0.15 | 0.12 | 0.39 | N.D | N.D. | N.D. |

1-41. (canceled)

42. An analog of humanized antibody Hu007 that specifically binds mature human IL-1β, wherein the analog comprises at least one amino acid substitution at positions 54, 55 or 56 of the heavy chain complementarity determining region 2 (CDR2),

| | |
|---|---|
| Glu Ile Leu Pro Xaa₅₄ Xaa₅₅ Xaa₅₆ Asn | (SEQ ID NO:1) |
| Ile Asn Tyr Asn Gln Lys Phe Lys Gly | |

wherein:
Xaa at position 54 is Gly, Ala, Ser, Val, Thr, Leu, Ile, Met, Phe, Tyr, or Trp;
Xaa at position 55 is Asn, Gln, Arg, Asp, Ser, Gly, or Ala; and,
Xaa at position 56 is Gly, Ala, Ser, Val, Thr, Leu, Ile, Met, Phe, Tyr, or Trp, provided that when Xaa₅₅ is Asn, then Xaa₅₆ is not Gly.

43. The analog of claim 42 wherein said amino acid substitution or substitutions reduces or eliminates deamidation at position 55 of the heavy chain CDR2 region.

44. The analog of claim 42 wherein Xaa₅₄ is Gly, Xaa₅₅ is Asn, and Xaa₅₆ is Val.

45. The analog of claim 42 wherein Xaa₅₄ is Gly, Xaa₅₅ is Asn, and Xaa₅₆ is Ala.

46. The analog of claim 42 wherein Xaa₅₄ is Gly, Xaa₅₅ is Asp, and Xaa₅₆ is Gly.

47. The analog of claim 42 wherein Xaa₅₄ is Gly, Xaa₅₅ is Gln, and Xaa₅₆ is Gly.

48. The analog of claim 42 wherein Xaa₅₄ is Gly, Xaa₅₅ is Ser, and Xaa₅₆ is Gly.

49. The analog of claim 42 wherein Xaa₅₄ is Gly, Xaa₅₅ is Ala, and Xaa₅₆ is Gly.

50. The analog of claim 42 wherein Xaa₅₄ is Gly, Xaa₅₅ is Gly, and Xaa₅₆ is Gly.

51. The analog of claim 42 wherein Xaa₅₄ is selected from the group consisting of Gly, Ala, Ser, Val, Thr, Leu, Ile, Met, Phe, Tyr, and Trp, Xaa₅₅ 55 is Ala, and Xaa₅₆ is Gly.

52. The analog of claim 42 wherein Xaa₅₄ is selected from the group consisting of Gly, Ala, Ser, Val, Thr, Leu, Ile, Met, Phe, Tyr, and Trp, Xaa₅₅ 55 is Gly, and Xaa₅₆ is Gly.

53. The analog of claim 42 wherein the analog comprises a light chain variable region having the sequence selected from the group consisting of SEQ ID NO:7 and SEQ ID NO:8.

54. The analog of claim 42 wherein the analog comprises a heavy chain variable region having the sequence selected from the group consisting of SEQ ID NO: 10 and SEQ ID NO:11.

55. The analog of claim 42, wherein the analog is: a) a single chain, b) a Fab fragment, or c) a F(ab′), fragment.

56. The analog of claim 42 wherein the analog has an IgG isotype selected from the group consisting of IgG1 and IgG4.

57. An isolated nucleic acid, comprising a polynucleotide encoding an analog of claim 42.

58. An expression vector comprising a nucleic acid according to claim 57.

59. A host cell stably transfected with the expression vector of claim 58.

60. A process for producing an antibody comprising culturing the host cell of claim 59 under conditions suitable for expression of said antibody and recovering said antibody from the cell culture.

61. The host cell of claim 59 wherein the host cell is selected from the group consisting of a Chinese Hamster Ovary cell, SP2/0 myeloma cell, NSO Myeloma cell, a syrian hamster ovary cell, and an embryonic kidney cell.

62. The host cell of claim 61 wherein said host cell is a Chinese Hamster Ovary cell.

63. A pharmaceutical composition comprising an analog of claim 42.

64. A method of treating rheumatoid arthritis or osteoarthritis, comprising administering to a subject an effective amount of the analog of claim 42.

65. A method of inhibiting the destruction of cartilage, comprising administering to a subject in need thereof an effective amount of the analog of claim 42.


SEQUENCE LISTING (12 sequences)

SEQ ID NO: 1 - protein, 17 aa, Artificial Sequence
  Synthetic Construct
EILPXXXNINYNQKFKG

SEQ ID NO: 2 - protein, 11 aa, Murine
KASQDIDRYLS

SEQ ID NO: 3 - protein, 7 aa, Murine
RVKRLVD

SEQ ID NO: 4 - protein, 8 aa, Murine
LQYDEFYT

SEQ ID NO: 5 - protein, 5 aa, Murine
RYWIE

SEQ ID NO: 6 - protein, 11 aa, Murine
IYYDYDQGFTY

SEQ ID NO: 7 - protein, 107 aa, Artificial Sequence
  Synthetic Construct
DIXMTQXPSSXXASXGXRVTITCKASQDIDRYLSWFQQKPGKAPKXLIYRVKRLVDGVPSRFSGSXSGXDYTLTISSLQPEDFATYYCLQYDEFPYTFGQGTKLEIK

SEQ ID NO: 8 - protein, 214 aa, Artificial Sequence
  Synthetic Construct
DIQMTQSPSSLSASVGDRVTITCKASQDIDRYLSWFQQKPGKAPKSLIYRVKRLVDGVPSRFSGSASGTDYTLTISSLQPEDFATYYCLQYDEFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 9 - protein, 22 aa, Artificial Sequence
  Synthetic Construct
MDMRTPAQFLGIFFFWFPGIRC

SEQ ID NO: 10 - protein, 120 aa, Artificial Sequence
  Synthetic Construct
XVQLVQSGAEVKKPGASVKVSCKXSGYTFXRYWIEWXRQAPGXGLEWXGEILPXXXNINYNEKFKGXXTXTADXSXXTAYMELSSLXSEDTAVYYCSTIYYDYDQGFTYWGQGTLVTVSS

SEQ ID NO: 11 - protein, 450 aa, Artificial Sequence
  Synthetic Construct
QVQLVQSGAEVKKPGASVKVSCKVSGYTFSRYWIEWVRQAPGKGLEWIGEILPGSGNINYNEKFKGKATITADTSTDTAYMELSSLRSEDTAVYYCSTIYYDYDQGFTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 19 aa, Artificial Sequence
  Synthetic Construct
MEWTWVFLFLLSVTAGVHS